(19) United States
(12) Patent Application Publication
(10) Pub. No.: US 2002/0160504 A1
(43) Pub. Date: Oct. 31, 2002

(54) Medium material selectively culturing prokaryote comprising rotting organic wastes and a method for cultivating crops using same

(76) Inventor: Jong-Uk Kim, Kyungsangbuk-do (KR)

Correspondence Address: Fulbright & Jaworski, 865 South Figueroa Street 29th Floor, Los Angeles, CA, 90017-2576, US

(21) Appl. No.: 09/958,754

(22) PCT Filed: Oct. 10, 2001
(86) PCT No.: PCT/KR01/00769

(30) Foreign Application Priority Data
May 12, 2000 (KR) 00-25321

(51) Int. Cl. C12N 1/20; C12N 1/00; C12N 5/02

(52) U.S. Cl. 435/253.6; 435/243; 435/420

(57) ABSTRACT

The present invention relates to a medium material for selectively culturing prokaryotes comprising rotting organic wastes and a method for cultivating crops using same. Specifically, the present invention provides a medium material for selectively culturing prokaryotes, which activates not only blue-green algae of Anabaena sp or Nostoc sp. but also photosynthetic bacteria of Rhodopseudomonas sp. or Rhodospirillum sp. in soil. According to the method for cultivating crops selectively culturing prokaryotes of the present invention, actually the method for cultivating crops selectively culturing prokaryotes is popularized over broad arable land, cultivating of crops using prokaryotes is possible by relatively low cost, an environment pollution is prevented from using an environment pollution material as raw materials for culturing prokaryotes, physicochemical characteristics of soil is improved, nourishments are continuously provided for crops, the growth of roots of crops is promoted, the efficiency of energy metabolism of crops increases and energy consumption dicreases, and disease resistance of crops improves and damage from disease and harmful insects decrease, thereby improving the quality and productivity of crops.

### BACKGROUND OF THE INVENTION

1. Field of the Invention

The present invention relates to a medium material for selectively culturing prokaryotes comprising rotting organic wastes and a method for cultivating crops using same, more specifically a medium material for selectively culturing prokaryotes, which activates not only blue-green algae of Anabaena sp. or Nostoc sp. but also photosynthetic bacteria of Rhodopseudomonas sp. or Rhodospirillum sp. in soil. Furthermore, the present invention also relates to a method for cultivating crops employing said medium material for selectively culturing prokaryotes to maintain and preserve optimal symbiotic relationship of effective soil microorganism, thereby improving the quality and productivity of crops, and crops cultivated by using the method.

2. Description of the Related Art

Since the beginning of agriculture by human kind, the agricultural technology had been little changed till now. Since 1960's, the constructions of large scale factory for chemical fertilizers and agricultural chemicals with industrial development had allowed farmers to abundantly use chemical fertilizer and agricultural chemicals, which are applicable easily and inexpensive, relatively, and government to recommend cultivation method using a plenty of fertilizers, therefore, the physicochemical characteristics of the soil were extremely aggravated, for example, all sorts of salts accumulation, agricultural chemicals remaining, soil acidification, the destruction of soil aggregate, etc. occurred, and at last it comes to be almost difficult to cultivate crops having high quality therein.

In order to recover the soil under deterioration to a good quality soil, many methods including fertilizing a plenty of organic fertilizers and using concentration-cultured soil microorganisms have been attempted. For examples, Korean Patent No. 2548 disclosed a method for producing nitrogen bacterial fertilizer, Korean Patent Application No. 97-13533 disclosed a fertilizer using sewage sludge and fly ash and a method for producing the same, Korean Patent Application No. 97-25148 disclosed a novel Bacillus genus bacterial strain and a method for promoting plant growth therewith, Korean Patent Application No. 98-21106 disclosed a complex powder of mixed microorganism and porous ore, Korean Patent Application No. 98-24315 disclosed an organic fertilizer for grass and flower using paper manufacturing sludge and a method for producing the same, Korean Patent Application No. 99-23238 disclosed a fermentation-producing substance culture soil for granule cultivation which improves fertilizer-efficacy and blue-green algae as fertilizers or soil enrichment agents, Japanese Patent Disclosure 4-108706, Japanese Patent Disclosure 5-194951, Japanese Patent Disclosure 6-16519, Japanese Patent Disclosure 6-80490, Japanese Patent Disclosure 7-118636, and Japanese Patent Disclosure 11-335191, etc. These disclosed methods using the extracts of blue-green algae or inputting blue-green algae mixed with auxiliary materials such as vermiculite, highly absorbent resin having biodegradability, or plastic vermiculite, etc, but having problems that using method spraying microorganisms comprising blue-green algae directly to soil caused excessive cost, other environmental contamination caused by auxiliary materials and the growth disorder of inputted microorganisms caused by native microorganisms, therefore, these methods were extremely inapplicable to actual arable land.

Furthermore, Korean Patent Application No. 96-033250 described soil enrichment agents made to adsorb Actinomyces, Rhizobium and photosynthetic bacteria to porous materials that allow to adsorb microorganisms abundantly, and a method for producing the same, but also having problem that comprising microbes in soil enrichment agent brings difficulties in production, storage and use of soil enrichment agent, and reduces its economical efficiency.

### SUMMARY OF THE INVENTION

The present invention is designed to resolve the problems of prior art as mentioned above, having an object to provide a medium material for selectively culturing prokaryotes to be more applicable in actual cultivated land, make cultivating crops by utilizing prokaryotes popularized at lower cost, and prevent environmental contamination effectively by employing organic wastes such as stock excretion, sludge, animal blood plasma, food wastes, etc, and fly ash etc., which are causal materials of environmental contamination, as raw materials for selectively cultivating prokaryotes.

Furthermore, the present invention has an object to provide a method for cultivating crops which can promote soil aggregation, biologically degrade salts accumulated in soil and making them effective to enhance a degree of effectiveness of nourishments for crops, furnish continuously nourishments to reduce amount of applied fertilizer, reduce amount of CO₂ and increase amount of O₂ in soil to facilitate the growth of crop root, minimize the energy consumption of crops by direct absorption of organic nutrients as organic form, enhance disease resistance of crops and reduce damages from disease and harmful insects, and maintain the freshness of crops and improve property of preservation thereof.

Still furthermore, the present invention has an object to provide crops which show the intrinsic properties of crops well and have a highly large content of calcium and minerals, their freshness lasts for long period to improve preservation, and particularly, in case of fruit, sugar content is high and scent is good.

In order to achieve said objectives, the present invention provides a medium material for selectively culturing prokaryotes produced by the mixed reaction of (a) 45 to 85 wt. % of rotting organic wastes comprising poultry excretion, (b) 5 to 35 wt. % of quick lime, (c) 1 to 10 wt. % of magnesia lime, and (d) 5 to 10 wt. % of zeolite, followed by drying the resultant product, which cultures selectively blue-green algae of Anabaena sp. or Nostoc sp. as well as photosynthetic bacteria of Rhodopseudomonas sp. or Rhodospirillum sp. in soil.

In addition, the present invention provides a method for cultivating crops selectively culturing prokaryotes, which is characterized by raising crops by selectively cultivating blue-green algae and photosynthetic bacteria from microorganisms in soil to make blue-green algae of Anabaena sp. or Nostoc sp. grow as dominant species in surface soil and make photosynthetic bacteria of Rhodopseudomonas sp or Rhodospirillum sp. grow as dominant species in subsurface soil.

Furthermore, the present invention provides crops cultivated according to said method.

### BRIEF DESCRIPTION OF THE DRAWINGS

FIG. 1 shows the position of blue-green algae and photosynthetic bacteria in biological world.

FIG. 2 shows the physicochemical change of products after reaction between rotting organic wastes and quick lime of the present invention.

FIG. 3 is a schematic perspective view showing a medium material reactor for selectively culturing prokaryotes according to the present invention.

FIG. 4 is a schematic sectional view of a medium material reactor for selectively culturing prokaryotes according to the present invention.

FIG. 5 is a partially detailed view of spraying means (A) of FIG. 4 according to the present invention.

FIGS. 6 & 7 are photographs showing red pepper cultivated according to examples of the present invention.

FIG. 8 is a photograph showing the appearance of greenhouse planting crops to compare bio farming method according to the present invention with organic farming method and chemical farming method of prior art.

FIG. 9 to FIG. 37 are photographs comparing crops cultivated by bio farming according to the present invention with crops cultivated by organic farming and chemical farming of prior art.

### DETAILED DESCRIPTION OF THE INVENTION

Hereinafter, the present invention will be explained in more detail.

The inventors have carried out numerous studies to solve problems generated when directly inputting microorganisms into soil, found that in case of employing rotting organic wastes causal materials of environmental contamination to make a material for selectively cultivating blue-green algae and photosynthetic bacteria, and selectively culturing prokaryotes already existed in arable land, for example, blue-green algae and photosynthetic bacteria to culture blue-green algae, particularly, Anabaena sp. or Nostoc sp. as dominant species in surface soil and photosynthetic bacteria, particularly, Rhodopseudomonas sp. or Rhodospirillum sp. as dominant species in subsurface soil, thereby cultivating crops, the problems are solved very efficiently, and come to complete the present invention.

Although a variety of microorganisms exist in soil, a medium material for selectively culturing prokaryotes of the present invention comprising rotting organic wastes performs function of promoting the activity of particularly blue-green algae and photosynthetic bacteria.

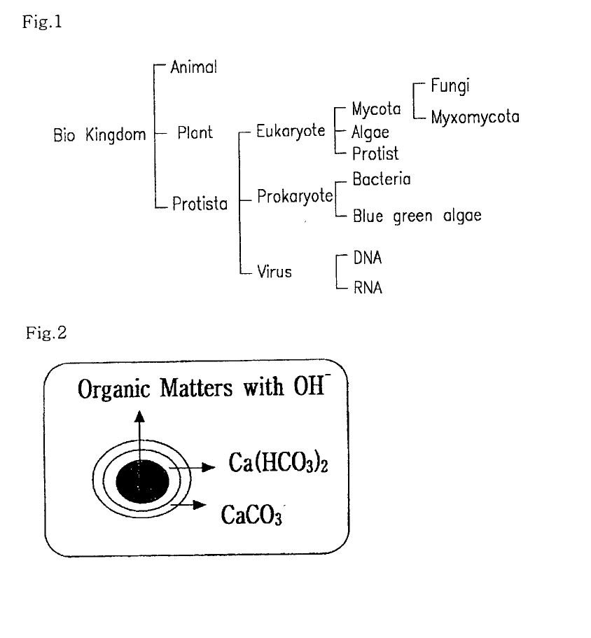

Blue-green algae (also named as Cyanobacteria) and photosynthetic bacteria are protophyte as shown in FIG. 1, and they grow autotrophically by utilizing sunlight even in the place where organic materials do not exist, only if moisture and light conditions are provided.

Blue-green algae of Anabaena sp. or Nostoc sp. and photosynthetic bacteria of Rhodopseudomonas sp. or Rhodospirillum sp. consist of useful materials such as protein, minerals, vitamin, carotenold, chlorophyll, fat, etc., and they independently grow by photosynthesis and also fix nitrogen in the air.

In addition, because they are very adaptable to environmental changes, Anabaena sp., Nostoc sp., Rhodopseudomonas sp., and Rhodospirillum sp. maintain moisture even at unfavorable condition, and search for the place which light necessary for photosynthesis is come into and grow to form colonies.

Anabaena sp. or Nostoc sp. are clustered just above or just below surface soil and photosynthesize using sunlight penetrating upper part of the layer, but they are also founded in the zone of 50 to 100 cm below the ground.

Photosynthetic bacteria are typically classified into green sulfur bacteria, purple sulfur bacteria and purple non-sulfur bacteria; however, the present invention selectively cultivates, particularly, purple non-sulfur bacteria of Rhodopseudomonas sp. or Rhodospirillum sp. from said photosynthetic bacteria as dominant species. Said Rhodopseudomonas sp. or Rhodospirillum sp. grows in the condition of pH and minerals equivalent to blue-green algae, being characterized by growing in anaerobic condition.

In order to selectively culturing blue-green algae of Anabaena sp. or Nostoc sp. and photosynthetic bacteria of Rhodopseudomonas sp. or Rhodospirillum sp., the present invention provides a medium material for selectively culturing prokaryotes produced by the mixed reaction of (a) 45 to 85 wt. % of rotting organic wastes comprising poultry excretion, (b) 5 to 35 wt. % of quick lime, (c) 1 to 10 wt. % of magnesia lime, and (d) 5 to 10 wt. % of zeolite, followed by drying the resultant product.

Said (a) rotting organic wastes comprising poultry excretion provide nourishments necessary for growing of prokaryotes. Said rotting organic wastes preferably contain 60 to 70 wt. % of moisture in consideration of the reaction with quick lime and the drying process. Furthermore, a medium material for selectively cultivating prokaryotes preferably contains 45 to 85 wt. %, more preferably 65 to 80 wt. %. Said poultry excretion contains numerous trace elements, and chicken excretion is most preferred due to its commercial availability.

Rotting organic wastes that can be used as mixture with poultry excretion are sludge, stock excretion, human excretion, animal blood plasma, food wastes, etc. For said stock excretion, pig excretion, dog excretion or all sorts of bred animal excretion are available. For said sludge, sludge generated from typical wastewater disposal plant is employed, and said animal blood plasma means animal bloods, being easily available from the place for butchering. For said food wastes, food or fish wastes discharged from restaurants are used after triturating. Said rotting organic wastes preferably comprise more than 50 wt. % of poultry excretion, most preferably 1-2:1 ratio of chicken excretion to sludge

Although said rotting organic wastes have been causal materials for water pollution and an offensive odor, the present invention utilizes said rotting organic wastes as useful sources used for growth of prokaryotes; therefore, the present invention are helpful to prevent environmental contamination.

Furthermore, said (b) quick lime (CaO) is preferably comprised in the medium material for selectively culturing prokaryotes, and quick lime having high activity is preferred.

In the medium material for selectively culturing prokaryotes of the present invention, rotting organic wastes comprising moisture with quick lime having high activity are mixed, and exothermic reaction between 120 to 200° C. when mixed; therefore, parasites and pathogens existed in rotting organic wastes are mostly sterilized, and the reaction between CaO and organic materials occurs to form organic compounds.

Furthermore, said magnesia lime is employed to introducing magnesium necessary for photosynthesis of chloroplast which is component of blue-green algae and photosynthetic bacteria, controlling ratio of calcium and magnesium of the medium material for selectively culturing prokaryotes to be 2.5 to 3.5:1. Said magnesia lime is preferably 1 to 10 wt. % of the medium material for selectively culturing prokaryotes.

Still furthermore, said medium material for selectively culturing prokaryotes comprises 6.5 to 10 wt. % of zeolite. In the medium material for selectively culturing prokaryotes of the present invention, said zeolite plays a role of enhancing stability and preservation properly of product and supplementing all sorts of trace elements. In addition, said zeolite plays a role of preventing losing useful materials such as physiological materials composed by prokaryotes, chelates combined with organic materials and trace elements, pyrrol or bilirubin, and improving the state of microorganisms.

In addition, the medium material for selectively culturing prokaryotes of the present invention may further comprise pulverized coal. Said pulverized coal is preferably 80 to 200 mesh as bark coal or sawdust, being produced by carbonizing bark or sawdust at 300 to 700° C. In case of comprising pulverized coal, the amount of said pulverized coal is preferably 0.1 to 5 wt. %. Pulverized coal has properties of alkalinity (pH 8 to 10) and porosity; therefore, when it is fertilized to soil with being comprised in the medium material for selectively culturing prokaryotes, heterotrophic microorganisms didn't survive in the pulverized coal, and only blue-green algae and photosynthetic bacteria survive, thereby helping to secure habitable space of blue-green algae and photosynthetic bacteria and further promoting to make blue-green algae and photosynthetic bacteria grow as dominant species.

Furthermore, the medium material for selectively culturing prokaryotes of the present invention may further comprise fly ash or submarine sludge for the balance of trace elements. Fly ash or submarine sludge contains evenly, zinc, aluminum, iron, manganese, etc., which are useful for growth of blue-green algae and photosynthetic bacteria. In case of comprising fly ash or submarine sludge, the amount is preferably 0.1 to 5 wt. % of the medium material for selectively culturing prokaryotes. Fly ash that can be uniformly mixed with other ingredients is more preferred.

The medium material for selectively culturing prokaryotes of the present invention is produced by the mixed reaction of (a) 45 to 85 wt. % of rotting organic wastes comprising poultry excretion, (b) 5 to 35 wt. % of quick lime, (c) 1 to 10 wt. %, of magnesia lime and (d) 5 to 10 wt. % of zeolite, followed by drying the resultant product, and furthermore, said reaction should be completed with uniformly mixing of all reactants and the reaction time is preferably 4 to 7 mins.

If adequate reaction is accomplished, the reaction as below formula I occurs, thereby forming product of organic materials coated with potassium bicarbonate and potassium carbonate. In the beginning of mixed reaction, quick lime captures organic materials and all sorts of minerals, by way of calcium hydroxide, forming calcium bicarbonate film in the form of surrounding organic materials and all sorts of minerals. A part of outer film of calcium bicarbonate film reacts again with carbon dioxide to form a film of calcium carbonate. In that case, if reaction time is too small and the mixture ratio of organic materials and quick lime is inadequate, incomplete materials is produced, not product as described in FIG. 2, and imperfect drying is achieved, and if reaction time is too large, the reaction between organic materials and quick lime are excessively achieved to be plaster; therefore, dissociation isn't well done when fertilized to soil, thereby being an obstacle to nutrient balance of soil and proliferation of prokaryotes.

[Formula I].

Organic materials+CaO+H₂O→Organic materials with OH⁻+Ca(OH)₂

Ca(OH)₂+2CO₂→Ca(HCO₃)₂

Ca(HCO₃)₂+CO₂→CaCO₃+H₂O+CO₂

For the sequence of combination, it is preferred to mix components of the medium material for selectively culturing prokaryotes except quick lime, and then mix and react quick lime. In case of mixing and react all components in one time or mixing rotting organic materials and quick lime in advance and then mixing the other components, there are problems that uniformly mixed reaction is not achieved and the period of drying is lengthened.

In order to control evenly mixed reaction and adequate reaction of said ingredients of raw material, a medium material reactor for selectively culturing prokaryotes is preferably used, which comprises a stirring portion where stirring of each ingredient of raw material is achieved, an oxygen inputting portion to blow in oxygen to the stirring portion, each hopper connected with the upper part of a stirring barrel to input rotting organic materials, magnesia lime, zeolite and quick time into the stirring barrel, and spraying means to spray quick lime inputted into the stirring barrel to the whole surface of the stirring barrel.

The medium material reactor for selectively culturing prokaryotes will be explained in detailed with reference to the attached drawings as follows:

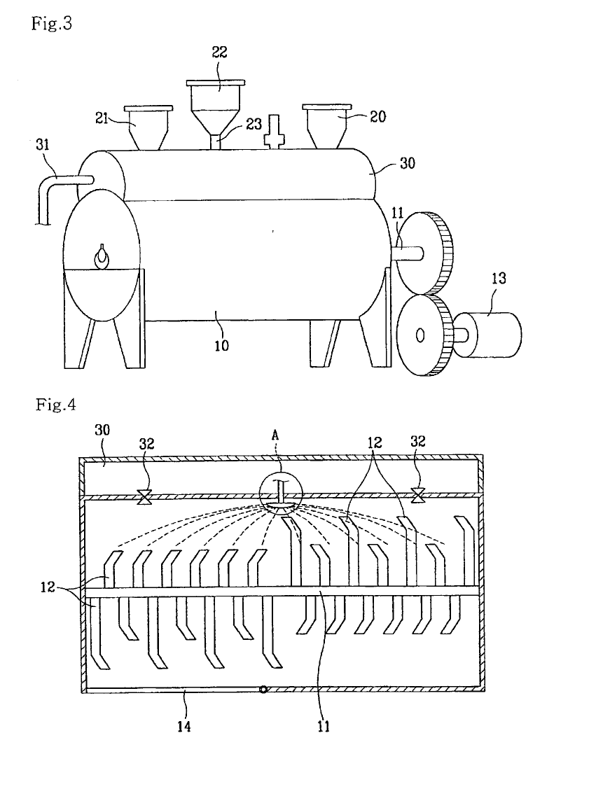

FIG. 3 is a schematic perspective view showing the medium material reactor for selectively culturing prokaryotes;

FIG. 4 is a schematic sectional view of the medium material reactor for selectively culturing prokaryotes; and

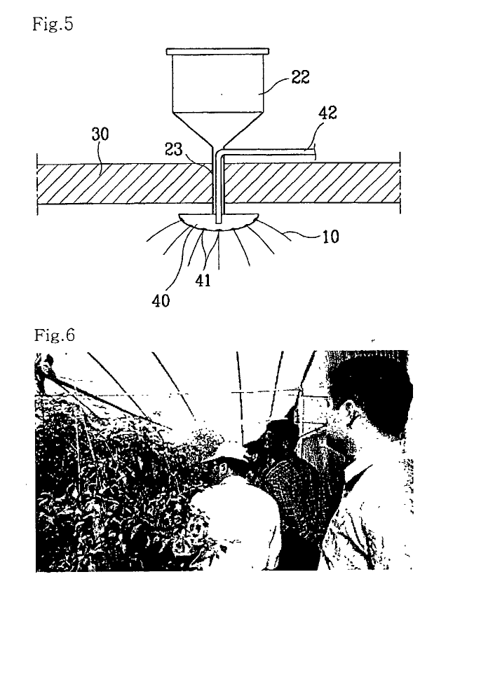

FIG. 5 is a partially detailed view of the spraying means (A) of FIG. 4.

According to the drawings as above-mentioned, said medium material reactor for selectively culturing prokaryotes is constituted by comprising a stirring barrel 10, a pivot 11 installed by passing through the center of the stirring barrel 10, stirring wings 12, in the inner portion of the stirring barrel 10, installed in a certain angle along the outer circumference surface of the pivot 11 and arranged in a longitudinal direction of the pivot 11, a driving motor 13 connected with the pivot 11 projected to an one sideline's end of the stirring barrel 10 to rotate the pivot 11, an oxygen inputting portion installed in one side of the stirring barrel 10 to blow in oxygen into the stirring barrel 10, each hopper 20, 21, 22 installed in the upper part of the stirring barrel 10 to input rotting organic materials, zeolite, magnesia lime and quick lime through a inputting pipe into the stirring barrel 10, and a spraying means A installed in a inputting pipe 23 of a hopper 22 for quick lime to spray quick lime to whole inner surface of the stirring barrel 10.

Here, other hoppers 20, 21 except the hopper for quick lime may be separately prepared to put in rotting organic materials, zeolite and magnesia lime respectively, or one hopper may be provided to put in premixed mixture of rotting organic materials, zeolite and magnesia lime.

Furthermore, said stirring barrel 10 of the medium material reactor for selectively culturing prokaryotes may be equipped with safety valves on one side surface of the barrel to exhaust gases generated from the inner portion when chemical reaction occurs, a pressure gauge, a thermometer etc. which may be annexed to confirm the reaction state, and a gate for exhaustion 14 provided in one side bottom surface to exhaust stirred contents.

Said stirring wings 12 is arranged in 180 degree of angle and continuously installed along the pivot 11. However, said the installed angle of the stirring wings 12 is not limited to 180 degree, 90 or 120 degree or any angle that can accomplish stirring makes no difference.

Furthermore, the stirring wings 12 has a structure that, to uniformly stir the center portion and inner circumference surface of the stirring barrel 10, one side stirring wing is elongated to the inner sideline end of stirring barrel and the other side stirring wing is relatively short to stir the center portion, and is symmetrically arranged and installed on the basis of center of the pivot 11.

Still Furthermore, the end of the stirring wings 12 has a structure of being folded in fixed angle to increase the stirring force.

Said oxygen inputting portion is constructed by comprising an air tank 30 installed at one side of the stirring barrel 10, a pump (not shown) to inject air through a supplying hose 31 connected to the air tank 30, and a check valve 30 installed between the air tank 30 and the stirring barrel 10 to input the air in the air tank 30 to the stirring barrel 10.

Therefore, if pressure of the air injected into the air tank 30 is exceed pressure in the stirring barrel 10, the air is inputted through the check valve 32 to enhance the stirring efficiency.

The influx of gas in the stirring barrel 10 to the air tank 30 is blocked because the check valve 32 is opened and operated only from air tank 30 to stirring barrel 10.

Meanwhile, said spraying means A is for spraying quick lime with the other reactant raw materials of rotting organic wastes, zeolite and magnesia lime, etc. to mix and react them, comprising a hemicycle type sprayer 40 located in the inner central upper portion of the stirring barrel 10 by connective installing at the lower end of the inputting pipe 23 which connects the hopper for quick lime 22 and the stirring barrel 10, a spraying nozzle for quick lime 41 formed along the curved surface of the sprayer 40, an air pipe 42 installed to sprayer 40 through the inside of the inputting pipe 23 to inject a compressed air, and a pump(not shown) for supplying the compressed air to the air pipe 42.

Therefore, by the compressed air, quick lime is sprayed through each nozzle 41, thereby being evenly sprayed to whole surface of the stirring barrel 10.

The function of said medium material reactor for selectively culturing prokaryotes will be explained as follow.

The mixture of rotting organic wastes, zeolite and magnesia lime is inputted into the stirring barrel 10 through the hoppers 20, 21, being mixed by rotating stirring wings 12. In case of producing the medium material for selectively culturing prokaryotes by comprising pulverized coal, fly ash or submarine sludge, in this step, pulverized coal, fly ash or submarine sludge are mixed together.

In other words, the pivot 11 connected to gear rotates at a certain velocity when the driving motor 13 rotates, and inputted mixture of rotting organic wastes, zeolite and magnesia is stirred while the stirring wings 12 installed at the pivot rotate in the stirring barrel 10.

At this time, each pump works to supply compressed air to the air tank 30 and the sprayer 40.

Compressed air supplied to the sprayer 40 through the air pipe 42 become to spray quick lime in a certain pressure through each nozzle 41 formed in the sprayer 40.

Meanwhile, the nozzles 41 formed in the sprayer 40 face separately different directions; therefore, quick lime is sprayed to separate directions through nozzles, being evenly sprayed over the whole surface of the stirring barrel 10.

Therefore, uniform chemical reaction over the entire reactants stirred is achieved by evenly spraying quick lime over the whole body of the stirring barrel 10,

On the other hand, the air supplied to the air tank 30 through the supplying hose 31 fills the air tank 30 at a certain pressure, being injected into the stirring barrel 10 through the check valve 32 where stirring is done.

The injection of the air into the stirring barrel 10 obtains the effect of promoting further chemical reaction by the oxygen contained in the air.

In this way, final medium material for selectively culturing prokaryotes is completed by chemical reaction by quick lime inputting, followed by drying process. The drying is preferably natural drying, not to change the components of material, and water content is preferably less than 35 wt. % in consideration of transportation and packaging.

The pH of medium material for selectively culturing prokaryotes of the present invention is preferably 10 to 12 in consideration of the pH preferred by blue-green algae and crops.

A method for cultivation crops using microorganisms or a method for soil enrichment so far has had an interest on only direct spraying of effective microorganisms, but these methods did not obtain satisfactory results despite large expense, that is, the effective microorganisms were not settled to be killed by resistance and competing relations of strong native microorganisms in spite of inputting a lot of effective microorganisms. However, the present invention does not spray microorganisms to soil, but fertilize to soil medium material for selectively culturing prokaryotes to activate synthetic native microorganisms of prokaryotes deactivated in arable land, such as Anabaena sp., Nostoc sp., Rhodopseudomonas sp. and Rhodospirillum sp, thereby reforming physical and chemical property of soil and accumulating nourishment sources to make the microorganisms state of soil the state which blue-green algae and photosynthetic bacteria are dominant species.

Furthermore, the present invention provides a method for cultivating crops selectively culturing prokaryotes, characterized by raising crops by selectively culturing blue-green algae of Anabaena sp. or Nostoc sp. and photosynthetic bacteria of Rhodopseudomonas sp. or Rhodospirillum sp. from microorganisms in soil to make blue-green algae of Anabaena sp. or Nostoc sp. grow as dominant species in surface soil and photosynthetic bacteria of Rhodopseudomonas sp. or Rhodospirillum sp. grow as dominant species in subsurface soil, preferably, the medium material for selectively culturing prokaryotes of the present invention is fertilized to soil of arable land, making blue-green algae of Anabaena sp. and Nostoc sp. and photosynthetic bacteria of Rhodopseudomonas sp. and Rhodospirillum sp. grow as dominant species.

Because blue-green algae of Anabaena sp. and Nostoc sp. and photosynthetic bacteria of Rhodopseudomonas sp. and Rhodospirillum sp. have equivalent growth condition except aerobic and anaerobic tendencies, by fertilizing the medium material for selectively culturing prokaryotes of the present invention, blue-green algae of Anabaena sp. or Nostoc sp. may be grown as dominant species under aerobic condition of surface soil and simultaneously, photosynthetic bacteria of Rhodopseudomonas sp. and Rodospirillum sp. may be grown as dominant species under anaerobic condition; the functions of oxidation and degradation of organic materials in soil may be inhibited, and an ideal synthetic soil for culturing crops may be completed.

In case that soil of arable land become dominated only by blue-green algae, blue-green algae responses very sensitively to the environment of cultivating, having problems that even with a little aggravation of state, for example, a sudden drop of temperature, etc., the system of soil microorganisms is changed by other harmful microorganisms; however, soil of which Anabaena sp. or Nostoc sp. are dominant species in surface and Rhodopseudomonas sp. and Rhodospirillum sp. are dominant species in subsurface has advantage that even if problem on cultivated environment arise transiently, the growth of Anabaena sp. or Nostoc sp. was promoted by the materials excreted by Rhodopseudomonas sp., or Rodospirillum sp., therefore, Anabaena sp. or Nostoc sp. become dominant species immediately again in surface soil. Especially, in our country where four seasons are distinguishable and therefore, the temperature of soil of arable land in the winter, because blue-green algae almost stop growing at less than 20° C., Rhodopseudomonas sp. or Rhodospirillum sp. stabilizes the environment of microorganisms of arable land soil, and functions complementally to again make the base for being dominant species of Anabaena sp. or Nostoc sp. in surface soil when cultivate crops in the next year. Furthermore, Rhodopseudomonas sp. or Rhodospirillum sp. has excellent ability of purification, for example, the removal of hydrogen sulfide or toxic amine in soil, etc., and the excretion of photosynthetic bacteria gives enhancing effect for the formation of flower and fruit of crops and enlargement of fruits.

In the mentioned above, crops comprise fruit trees, vegetables and cereals, and any plant that human beings cultivate with sense of purpose will be comprised. Of course, said crops comprise medicinal plants, grass, etc.

The medium material for selectively culturing prokaryotes comprising rotting organic wastes of the present invention proliferates soil prokaryotes, particularly, Anabaena sp. or Nostoc sp, Rhodopseudomonas sp. or Rhodospirillum sp. to convert soil to synthetic soil. Arable land converted to synthetic soil according to the medium material for selectively culturing prokaryotes comprising rotting organic wastes of the present invention produces functional crops high in the content of calcium and minerals by strong chelate action not shown in degradable soil which heterotrophic microorganisms are dominant species.

A method for cultivating crops comprising culturing blue-green algae of the present invention, for rice filed soil, preferably makes Anabaena sp. or Nostoc sp. grow as dominant species in a zone of 10 to 15 cm from surface soil, and Rhodopseudomonas sp. or Rhodospirillum sp. grow as dominant species in soil below the zone. Furthermore, in dry field, Anabaena sp. or Nostoc sp. are preferably grown as dominant species in a zone of 2 to 5 cm from surface soil, and photosynthetic bacteria of Rhodopseudomonas sp. or Rhodospirillum sp. are preferably grown as dominant species in soil below the zone. More preferably, the growth of actinomicetes is controlled by repeating drying and moisturizing dry field periodically, which gives soil fungistasis to reduce damages from disease and harmful insects.

In cultivating crops, the amount of applied medium material for selectively culturing prokaryote is appropriately controlled according to crops. For rice farming, total 2 times, 60 to 100 kg/1 time/10 a of fertilizing as additional manuring is preferred, and for dry filed, 200 to 300 kg/10 a of fertilizing as basal dressing is preferred. More preferably, vegetables are fertilized with 1 kg/3.3 m² (also called as pyong in Korea) after rotary work, flowering plants are supplied with sufficient moisture after 1 kg/pyong of fertilizing, for grass, 1 kg/pyong of respectively fertilizing in the early spring and late fall, that is, 2 times per year, are preferred, and fruit plants are respectively fertilized with 1 kg/pyong when plants bud and after harvest, that is, 2 times per year.

Furthermore, correcting pH of soil by 7.0 to 7.5 is preferred before fertilization of the medium material for selectively culturing prokaryotes, and in dry field of sandy soil where has strong acidity, larger amount of the medium material for selectively culturing prokaryotes are preferably fertilized. In using the medium material for selectively culturing prokaryotes comprising rotting organic wastes of the present invention, using the material together with immature compost is not preferred due to a risk of gas hindrance in the early of crops growth.

Still furthermore, blue-green algae of Anabaena sp. or Nostoc sp. and photosynthetic bacteria of Rhodopseudomonas sp. or Rhodospirillum sp. is promoted to grow rapidly if supply of effective moisture increase; on the contrary, if supply of moisture is so insufficient that extreme change of moisture is happened, metabolism of microorganisms become confused. Especially, blue-green algae decrease rapidly. Therefore, in case of cultivating crops by fertilizing the medium material for selectively culturing prokaryotes comprising rotting organic wastes of the invention, if possible, maintaining soil as extra-moisturized state (more than 40% of soil moisture content) is preferred.

In addition, the method for cultivating crops selectively culturing prokaryote of the present invention restores to soil, organic compounds coated with calcium carbonate by chemical reaction, thereby not creating the generation of bad smell due to rapid degradation, gas hindrance, etc., supplies organic materials continuously by Anabaena sp., Nostoc sp., Rhodopseudomonas sp., or Rhodospirillum sp., thereby having fertilizer efficiency till the latter part of crops growth, and chelates components of soil accumulated with salts by proliferation of microorganisms to prevent direct reaction with other ingredients of soil, thereby making absorption of the accumulated salts as nutrients source to crops

Furthermore, the present invention provides crops cultivated by the method for cultivating crops selectively culturing prokaryotes, said crops have characteristics of their intrinsic properties largely enhanced. Especially, the crops cultivated by the present invention contain high calcium and mineral content. For example, rice plant has large grain and the cooked rice made from said rice was remarkably tasty and more glossy, and had high sweetness, compared with the cooked rice made form traditionally cultivated rice; in case of fruit plants, sugar content, flavor, taste, and size of fruits produced from said fruit plants are greatly enhanced to improve the quality of products; and in case of vegetables, their flesh are soft flesh but felt crisping, size, flavor, color and preservation property of products are remarkably enhanced to improve the quality of products.

Hereinafter, the examples of the present invention will be described. However, these are to illustrate the present invention, and the present invention is not limited thereto.

### EXAMPLE 1

By using the medium material reactor for selectively culturing prokaryotes, 800 kg of chicken excretion containing 65 wt. % of moisture as rotting organic wastes, 50 kg of magnesia lime, and 70 kg of zeolite were mixed and then, 200 kg of quick lime were mixed with the mixture and reacted for 5 mins., followed by drying the reactant to prepare the medium material for selectively culturing prokaryotes having 30 wt. % of water content ratio.

### EXAMPLE 2

The medium material for selectively culturing prokaryotes was prepared by the method of Example 1, except that 500 kg of chicken excretion and 250 kg of sludge were used as rotting organic wastes.

### EXAMPLE 3

The medium material for selectively culturing prokaryotes was prepared by the method of Example 1, except that 20 kg of pulverized coal were further comprised as raw materials ingredients.

### EXAMPLE 4

The medium material for selectively culturing prokaryotes was prepared by the method of Example 1, except that 30 kg of fly ash were further comprised.

### EXAMPLE 5

The medium material for selectively culturing prokaryotes was prepared by the method of Example 2, except that 500 kg of chicken excretion and 250 kg of pig excretion were used as rotting organic wastes.

### EXAMPLE 6

The medium material for selectively culturing prokaryotes was prepared by the method of Example 2, except that 500 kg of chicken excretion and 250 kg of human excretion were used as rotting organic wastes.

### EXAMPLE 7

The medium material for selectively culturing prokaryotes was prepared by the method of Example 2, except that 500 kg of chicken excretion and 20 kg of pig blood plasma were used as rotting organic wastes.

### EXAMPLE 8

The medium material for selectively culturing prokaryotes was prepared by the method of Example 2, except that 500 kg of chicken excretion and 50 kg of chopped fishes were used as rotting organic wastes.

### EXAMPLE 9

The medium material for selectively culturing prokaryotes was prepared by the method of Example 2, except that 500 kg of chicken excretion and 100 kg of residual meals were used as rotting organic wastes.

### EXAMPLE 10

The medium material for selectively culturing prokaryotes was prepared by the method of Example 1, except that 500 kg of chicken excretion, 250 kg of sludge, 20 kg of pulverized coal and 30 kg of fly ash were used as rotting organic wastes.

### COMPARATIVE 1

In the medium material reactor for selectively culturing prokaryotes, 500 kg of chicken excretion and 200 kg of quick lime were mixed and reacted for 5 mins., followed by drying the reactant to prepare a material having 30 wt. % of water content ratio.

### COMPARATIVE 2

The material was prepared by the method of Example 1, except 20 hrs. of reaction time.

### COMPARATIVE 3

The material was prepared by the method of Example 1, except that 800 kg of pig excretion instead of chicken excretion were used.

[Experiment] 1

Selectively Culturing Prokaryotes

The materials prepared in the Example 1 to 10 and Comparative 1 to 3 were sprayed to soil collected from 4 places of arable land, all parts of the country, and the extent of selectively culturing prokaryotes was observed. Specific locations of the arable lands collected are described below.

Arable land 1: 33-187 woonjeon-li, mokcheon-myon, cheonan-si, choongcheongnam-do, Korea.

Arable land 2: 121 songwon-dong, naju-si, jeollanam-do, Korea,

Arable land 3: 316 hwengkye1-li, doam-myon, pyeongchang-gun, gangwon-do, Korea.

Arable land 4: 477-7 iho-li, gangcheon-myon, yeoju-gun, gyeonggi-do, Korea.

The collected soil filled each port to 30 cm in height, which is 30 cm in width, 30 cm in length and 40 cm in height, and then, materials prepared in the Example 1 to 10 and Comparative 1 to 3 were fertilized to soil by 100 g, and the moisture of soil was kept at more than 40 wt. % by frequently supplying water. Furthermore, as comparative experiment, a case that nothing was fertilized and no water was supplied was called as blank test-1, and a case that nothing was fertilized and only water was controlled was called as blank test-2. For each port, the state of soil for 1 month after fertilization was observed.

As results of observation, when microorganisms comprising total prokaryotes were quantitatively estimated, the number of microorganisms was slightly decreased in blank test-1, and the same level as that of initial number in blank test-2. In case that materials of the Example 1 to 10 of the present invention were used, the number of microorganisms were explosively increased immediately after inputting, and the velocity of increasing was particularly high in the order of using material of the Example 10>Example 3>Example 4 and cases of the Example 1 & 2 and the Example 5 to 9 show equal velocity.

On the contrary, although the Comparative 1 and Comparative 3 show equivalent velocity of microorganisms increase, the level did not noticeably come up to that of the Example 1. In the Comparative 2, the number of microorganisms did not almost increase in the early stages, and increase after 2 weeks, not coming up to that of the Comparative 1.

In using materials of the Example 1 to 10, photosynthetic bacteria increased explosively immediately after inputting, and blue-green algae, of which Anabaena sp., and Nostoc sp. were major species, became dominant species in a zone of 2 to 5 cm from surface soil after about 7 to 9 days (7 days in case of the Example 10, and 9 days in case of the Example 5 to 9) and in soil below the zone, photosynthetic bacteria of Rhodopseudomonas sp., or Rhodospirillum sp. became dominant species.

### EXAMPLE 11

### Cultivation of Rice Plants

When the aimed number of leaves (20 to 22) was secured, 100 kg/10 a of the medium material for selectively culturing prokaryotes prepared according to the Example 1 were sprayed to rice plants. After fertilization, rice plants were maintained at water-containing condition without removing water, and the pH of contained water was controlled to approximately 7.5. When right after fertilizing the medium material for selectively culturing prokaryotes, photosynthetic bacteria increased largely, and when about 10 days elapsed right after fertilizing the material, red blue-green algae became generated, and after 1 month, they turned into green. At this time, soil microorganisms become dominated by Anabaena sp., and Nostoc sp. in a zone of 12 cm from surface soil, and in soil below the zone, photosynthetic bacteria of Rhodopseudomonas sp., or Rhodospirillum sp. became dominant species, largely containing lactate-producing bacteria.

Although rice plants are acidophilic crops that grow well in weak acidic soil, they grow better in weak alkaline environment at the latter period during growth. If the medium material for selectively culturing prokaryotes is sprayed when the aimed tillering number of 20 to 22 is secured, the tillering of rice plant is stopped. At this time, effective tillers gradually turn into deep green color and their mesophylls became large and strong. In general farming, withering of low leaves often occurs in ripening season, while in the cultivation method of the present invention, little withering occurred. In addition, it was observed in the roots of rice plants that thick white roots strongly extended. This results from the fact that the increase of amount of supplied oxygen by blue-green algae and photosynthetic bacteria helps the growth of root. Phanerophyte gradually colored deep green and growing strongly, and thus, little rice failing occurred, due to typhoon and heavy rain. In addition, surrounding leaves felt soft but tough and strong.

The yield of the rice cultivated according to the present invention was 640 kg based on 10 a, and the rice cultivated according to the present invention had larger grain and was more glossy, compared with traditionally cultivated rice and the cooked rice made from said rice was sticky and had high sweetness.

### EXAMPLE 12

### Cultivation of Cucumbers

To the area of cultivation house of 300 pyong, i.e., 10 a., 300 kg of the medium material for selectively cultivating prokaryotes prepared in the Example 2 were fertilized, followed by mulching, and high moisture state, i.e., the extent of soil water content 50 wt. %, was maintained. When 20 days elapsed after fertilization of the medium material for selectively culturing prokaryotes, seedling trees were planted. At this time, soil microorganisms were dominated by blue-green algae of Anabaena sp., and Nostoc sp. in a zone of 3 cm from surface soil, and in soil below the zone, photosynthetic bacteria of Rhodopseudomonas sp., or Rhodospirillum sp. were dominant species.

Seeds were rooted about 4 days after planting, and the growth condition became favorable and they grew vigorously. In addition, in order to prevent damages from disease and harmful insects, the mixture of trees and grass liquid and chitosan was sprayed to surface of leaves about 2 times per week. For about 4 months from 40 days after planting, approximately 200,000 kg were harvested. The grown states were satisfactory, and the ratio of fruit falling did not come up to even 0.1%. The produced cucumber had soft flesh but crisping texture and unique flavor and taste. Especially, unlike traditionally cultivated cucumbers, the produced cucumber had not bitter taste even at end portion. During growth, no physiological hindrance occurred.

### EXAMPLE 13

### Cultivation of Tomatoes

2 weeks before planting, 200 kg of the medium material for selectively cultivating prokaryotes prepared in the Example 10 were fertilized to 10 a as basal dressing, followed by enough irrigation. Punching was conducted after mulching, and planting was conducted on Mar. 15, 2000.

At this time, soil microorganisms were dominated by blue-green algae of Anabaena sp., and Nostoc sp. in a zone of 3 cm from surface soil, and in soil below the zone, photosynthetic bacteria of Rhodopseudomonas sp., or Rhodospirillum sp. were dominant species, largely containing actinomycetes. The harvest was conducted from May to the middle of August, and big fruits were uniformly grown in trees in spite of raising no fruits to 4 stages. The color of leaves was deeper green than those cultivated by traditional farming method, and particularly, the color became remarkably deep at harvesting period.

The tomatoes cultivated in this Example had sugar content near 9 and had unique favor of tomato, and had big size and flesh. And, if the tomatoes were immersed in water, most of them were sunk down. This proved that fruit having hollow did not be produced. In addition, fully ripen fruits were harvested, and they maintained freshness even when 5 to 6 days elapsed after harvest.

### EXAMPLE 14

### Cultivation of Red Peppers

To the area of cultivation house of 300 pyong, i.e., 10 a., 250 kg of the medium material for selectively cultivating prokaryotes prepared in the Example 1 were fertilized, followed by mulching, and high moisture state, i.e., the extent of soil water content 40 wt %, was maintained. When 20 days elapsed after fertilization of the medium material for selectively culturing prokaryotes, seedling trees were planted. At this time, soil microorganisms were dominated by blue-green algae of Anabaena sp., and Nostoc sp. in the zone of 3.5 cm from surface soil, and in soil below the zone, photosynthetic bacteria of Rhodopseudomonas sp., or Rhodospirillum sp. were dominant species.

Seeds of red pepper were grown to the extent of 110 to 130 cm if cultivated by traditional method of cultivation, but the seeds cultivated according to the present invention were grown more than 2 m as shown in FIG. 6, and the red pepper had concentrated deep green color and straight shape, in addition, the harvest increased to the extent of 40%, compared with traditional method of cultivation.

[Experiment] 2

Comparison with Other Methods of Cultivation

To compare the case of cultivating crops by the method for cultivation crops selectively culturing prokaryotes of the present invention with the case of cultivating crops by traditional method of cultivation, i.e., organic farming method and chemical farming method, cultivation house was equally divided

To the area of cultivation house of 300 pyong, i.e., 10 a., 250 kg of the medium material for selectively cultivating prokaryotes prepared in the Example 1 were fertilized, followed by mulching, and high moisture state, i.e., the extent of soil water content 40 wt. %, was maintained. When 20 days elapsed after fertilization of the medium material for selectively culturing prokaryotes, seedling trees were planted. At this time, soil microorganisms were dominated by blue-green algae of Anabaena sp., and Nostoc sp. in the zone of 3.5 cm from surface soil, and in soil below the zone, photosynthetic bacteria of Rhodopseudomonas sp., or Rhodospirillum sp. were dominant species.

Seeds of red pepper were grown to the extent of 110 to 130 cm if cultivated by traditional method of cultivation, but the seeds cultivated according to the present invention were grown more than 2 m as shown in FIG. 6, and the red pepper had concentrated deep green color and straight shape, in addition, the harvest increased to the extent of 40%, compared with traditional method of cultivation.

[Experiment] 2

Comparison with Other Methods of Cultivation

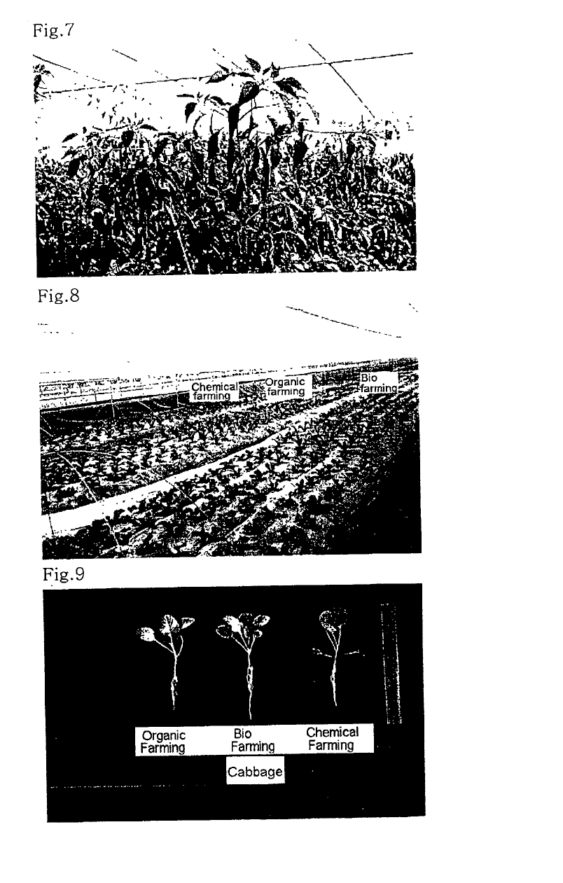

To compare the case of cultivating crops by the method for cultivation crops selectively culturing prokaryotes of the present invention with the case of cultivating crops by traditional method of cultivation, i.e., organic farming method and chemical farming method, cultivation house was equally divided and crops were planted as shown in FIG. 8.

The crops were cabbage, kale, lettuce, red mustard, Brassica campestris, pak-choi, radish, Raphanus sativus L., red chard, perilla, caesar green, shallot, red chicory, spinach, red leaf mustard, okra, dark red curled leaf, lollo, spring cabbage, Brassica juncia L., chinese leek, beet, carrot, Raphanus raphanistrum, red onion, welsh onion, chicory, caesar red, or red curled leaf, and planting was conducted 2 weeks after fertilization of basal dressing.

In the bio farming of the present invention, 300 kg of the medium material selectively cultivating prokaryotes were fertilized to 300 pyong of the area; in the chemical farming, the composite fertilizer of Nam Hae Chemicals comprising nitrogen, phosphate and potassium was fertilized as basal dressing and additional manuring according to the recommended amount; and in the organic farming, 2,000 kg of matured compost were fertilized to 300 pyong of the area as basal dressing. Soil moisture was kept to be more than 40 wt. % by irrigating between furrows as in the FIG. 8.

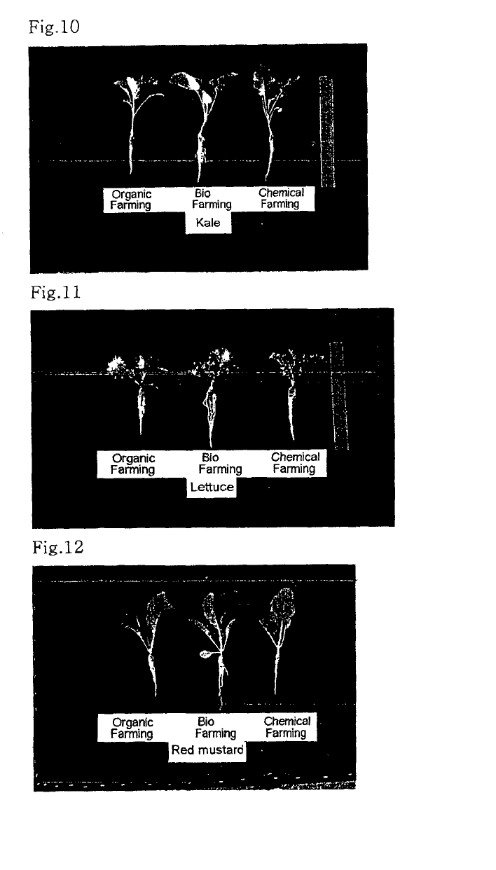

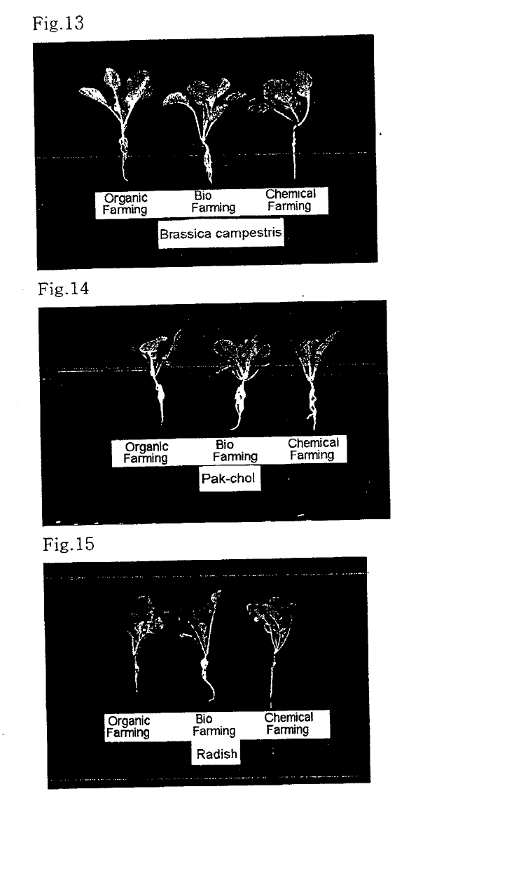

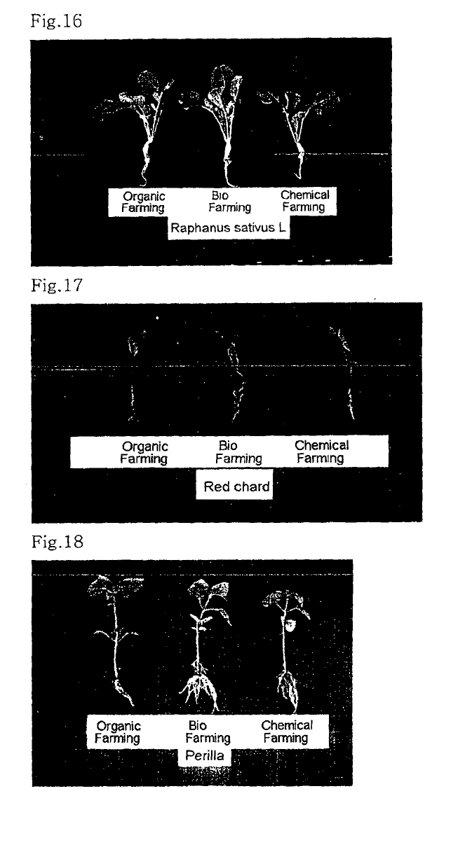

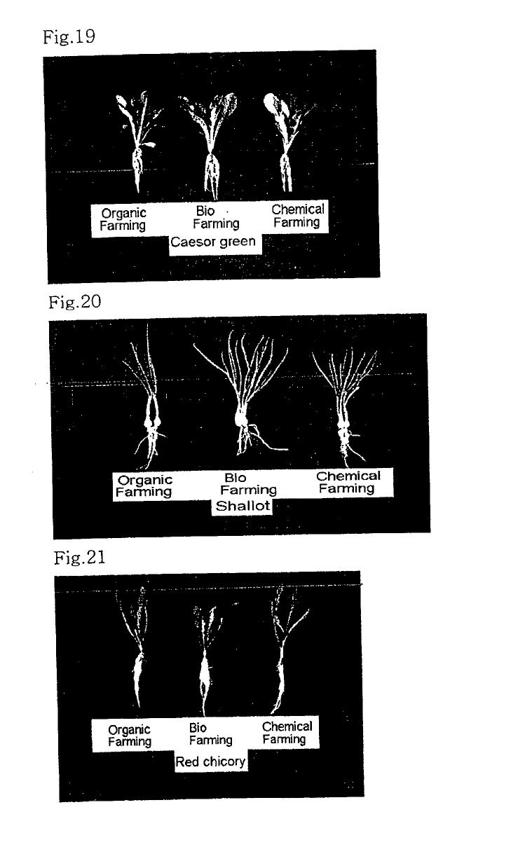

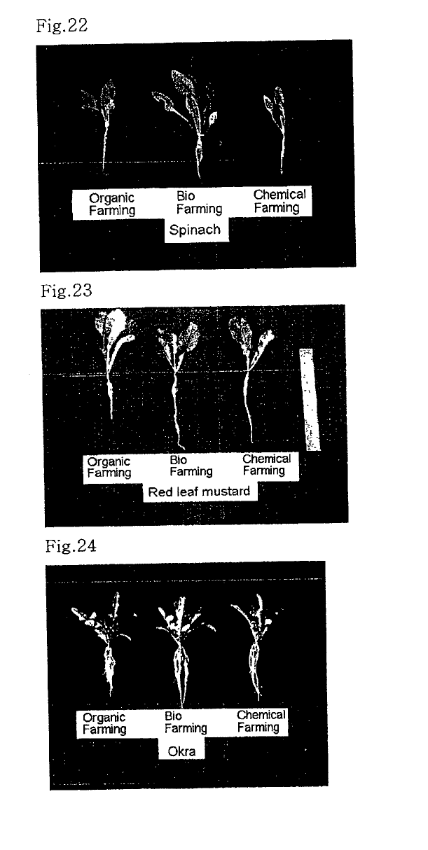

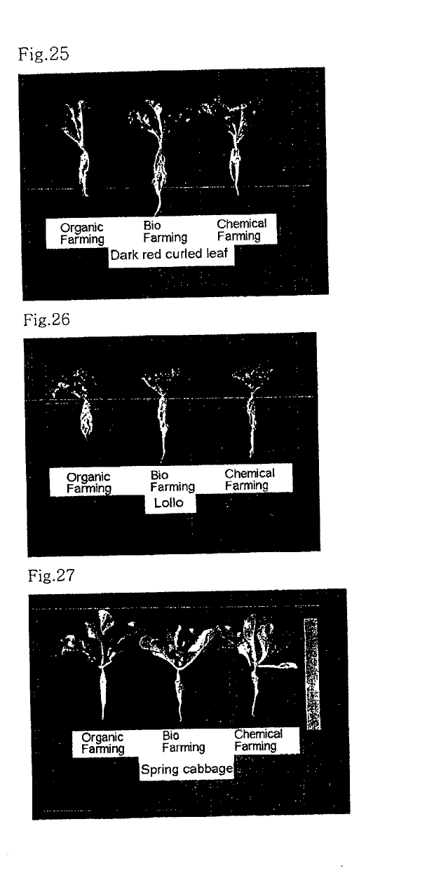

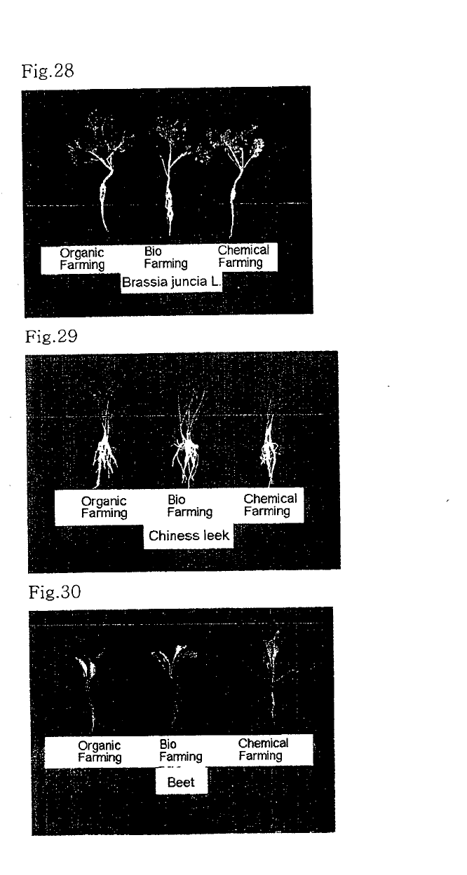

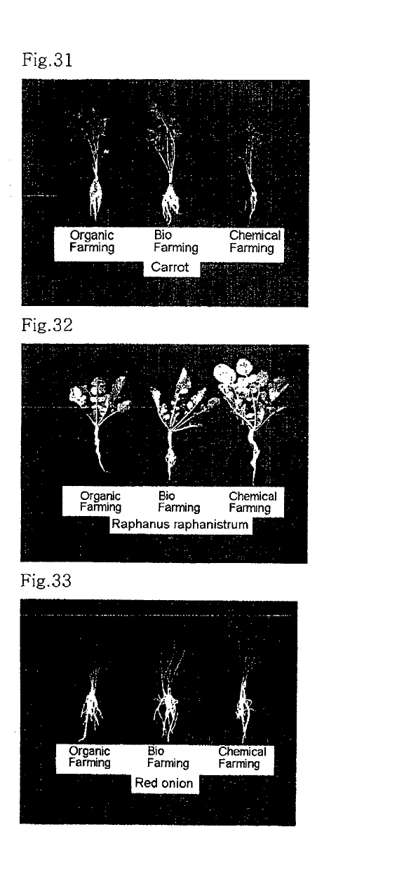

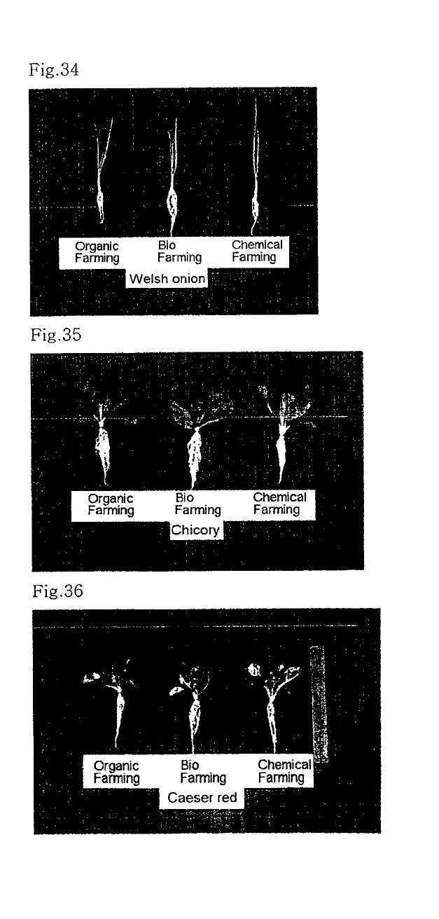

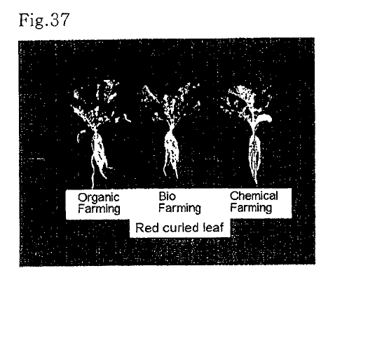

When each crop grew initially to a certain extent, each crop cultivated by each farming method was sampled, and compared with each other. FIG. 9 to FIG. 37 show crops cultivated by each farming method with photographs.

As shown in the FIG. 9 to FIG. 37, the crops cultivated by bio farming according to the present invention developed root growth and foliage, particularly, root growth, compared with the crops cultivated by organic farming method or chemical farming method. In addition, the crops cultivated by the bio farming method according to the present invention had intrinsic color and scent of the crops strongly.

Furthermore, in case of chemical farming method, only crops around furrows irrigated grew large and the crops of the center region mulched did not grow well, and in case of organic farming method, crops grew more uniformly than in case of chemical farming method, but noticeably did not come up to the case of bio farming. The crops cultivated according to the present invention grew uniformly and very well over the whole mulched portion, and in the soil cultivated by organic farming or chemical farming method, invisible various kinds of weeds were generated. This is understood as follows: in case of bio farming method, the seeds dormant in soil come to life due to continuously supplying nourishments to soil, and physiological active materials excreted and the improvement of plant growth condition by prokaryotes.

Furthermore, as time goes on to the latter part of the crop growth, the difference between bio farming method and other farming methods was more remarkable, and from the aspects of quantity and quality in harvest, bio farming method was so excellent that it was incomparable with chemical farming method.

As mentioned above, the medium material for selectively cultivating prokaryotes comprising rotting organic wastes of the present invention can

What is claimed is:

1. A medium material for selectively culturing prokaryotes produced by the mixed reaction of (a) 45 to 85 wt. % of rotting organic wastes comprising poultry excretion, (b) 5 to 35 wt. % of quick lime, (c) 1 to 10 wt. % of magnesia lime, and (d) 5 to 10 wt. % of zeolite, followed by drying the resultant product, which cultures selectively blue-green algae of Anabaena sp. or Nostoc sp. as well as photosynthetic bacteria of Rhodopseudomonas sp. or Rhodospirillum sp. in soil.

2. A medium material for selectively culturing prokaryotes according to claim 1, further comprising 0.1 to 5 wt. % of pulverized coal.

3. A medium material for selectively culturing prokaryotes according to claim 1, further comprising 0.1 to 5 wt. % of fly ash or submarine sludge.

4. A medium material for selectively culturing prokaryotes according to claim 1, wherein said (a) poultry excretion is chicken excretion.

5. A medium material for selectively culturing prokaryotes according to claim 1, wherein said (a) rotting organic wastes comprise 1-2:1 wt. ratio of chicken excretion to sludge.

6. A medium material for selectively culturing prokaryotes according to claim 1, wherein said mixed reaction is proceeded for 4 to 7 mins.

7. A medium material for selectively culturing prokaryotes according to claim 1, wherein said mixed reaction is accomplished in a medium material reactor for selectively culturing prokaryotes, comprising a stirring barrel, a pivot installed by passing through the center of the stirring barrel, stirring wings, in the inner portion of stirring barrel, installed in a certain angle along the outer circumference surface of the pivot and arranged in a longitudinal direction of the pivot, a driving motor connected with pivot projected to an one sideline's end of the stirring barrel to rotate the pivot, an oxygen inputting portion installed in one side of the stirring barrel to blow in oxygen into the stirring barrel, each hopper installed in the upper part of stirring barrel to input rotting organic materials, zeolite, magnesia lime and quick lime through a inputting pipe into the stirring barrel, and a spraying means installed in the inputting pipe of a hopper for quick lime to spray quick lime to whole inner surface of stirring barrel.

8. A medium material for selectively culturing prokaryotes according to claim 7, wherein said stirring wings have a structure that stirring wings are arranged in 180 degree of angle, one side stirring wing is elongated to the inner sideline end of stirring and the other side stirring wing is relatively short to stir the inner circumference portion and the center portion of stirring barrel, and the end of the stirring wings are folded in fixed angle.

9. A medium material for selectively culturing prokaryotes according to claim 7, wherein said oxygen inputting portion comprises an air tank installed at one side of stirring barrel, a pump to inject air through a supplying hose connected to the air tank, and a check valve installed between the air tank and the stirring barrel to input the air in the air tank to the stirring barrel.

10. A medium material for selectively culturing prokaryotes according to claim 7, wherein said spraying means comprises a hemicycle type sprayer located in the inner central upper portion of stirring barrel by connective installing at the lower end of an inputting pipe which connects a hopper for quick lime and the stirring barrel, a spraying nozzle for quick lime formed along the curved surface of sprayer, an air pipe installed to the sprayer through the inside of the inputting pipe to inject a compressed air, and a pump for supplying the compressed air to the air pipe.

11. A method for cultivating crops selectively culturing prokaryotes, wherein said crops are cultivated by selectively culturing blue-green algae and photosynthetic bacteria from microorganisms in soil, thereby making blue-green algae of Anabaena sp. or Nostoc sp. grow as dominant species in surface soil and photosynthetic bacteria of Rhodopseudomonas sp. or Rhodospirillum sp. grow as dominant species in subsurface soil.

12. A method for cultivating crops selectively culturing prokaryotes according to claim 11, characterized in that a medium material for selectively culturing prokaryotes according to any of claims 1 to 11 is fertilized to soil of arable land.

13. A method for cultivating crops selectively culturing prokaryotes according to claim 11, wherein moisture of said soil is maintained to more than 40 wt. %.
